(19) United States
(12) Patent Application Publication
(10) Pub. No.: US 2002/0128610 A1
(43) Pub. Date: Sep. 12, 2002

(54) Pen syringe needle adapter

(76) Inventor: Spencer H. Owades, Haiku, HI (US)

Correspondence Address: Spencer H. Owades, 1041 Kauhikoa Road, Haiku, HI, 96708, US

(21) Appl. No.: 10/029,959

(22) Filed: Dec. 21, 2001

Related U.S. Application Data
(60) Provisional application No. 60/257,256, filed on Dec. 22, 2000.

(51) Int. Cl. A61M 5/00

(52) U.S. Cl. 604/241

(57) ABSTRACT

The invention described here is an adapter that enables the use of different kinds of double-ended needles on a pen style medication delivery syringe.

References Cited:

| | | |
|---|---|---|
| 5,984,906 | Nov. 1999 | Bonnichsen, et al |
| 6,001,089 | Dec, 1999 | Burrroughs, et al |
| 6,146,361 | Nov, 2000 | DiBiasi, et al |

### SPECIFICATION

### BACKGROUND

1. Field of Invention

The invention relates to self-administered medication delivery systems. Otherwise known as pen-style syringes, these consist of a pre-filled medicine reservoir, a dose-controlling mechanism, a piston or plunger and provision for attachment of a hypodermic needle at the distal end.

Specifically, this invention relates to the attachment mechanism of the hypodermic needle to the pen style syringe.

2. Description of Prior Art

The pen style syringe has gained wide acceptance among insulin-dependent diabetics because of the accuracy of dosing, convenience of use and recyclable nature of the device. (See U.S. Pat. Nos. 5,984,906 and 6,001,089).

The pen syringe's major disadvantage is the limited choice in size and high cost of disposable needles to affix to the syringe. (See U.S. Pat. No. 6,146,361)

There has been much discussion about pain perception when self-administering medication, and how the size of the needle affects this perceived pain. Needle diameter is directly proportional to the insertion pressure of the needle; lumen diameter is inversely proportional to the injection pressure required to deliver the medication dose. Hence, thinner needles may seem less panful to insert, but may seem more painful when medication is expressed through them into the tissue. Clearly, there is a balance between the two, and it is advantageous to the consumer to make this choice.

### OBJECTS AND ADVANTAGES

The object of the invention is to enable the user of pen style syringes a wider choice of disposable needles with which to self-administer their medication.

The advantage of the invention is greater freedom of choice in needle selection, and lower cost to the consumer.

### DRAWING FIGURES

FIG. 1 shows a longitudinal cross-section of the invention

FIG. 2 shows the application of the invention to a pen style syringe

FIG. 3 shows the application of a standard, double-ended dental needle to the invention

FIG. 4 shows the complete assembly of pen syringe, invention and needle

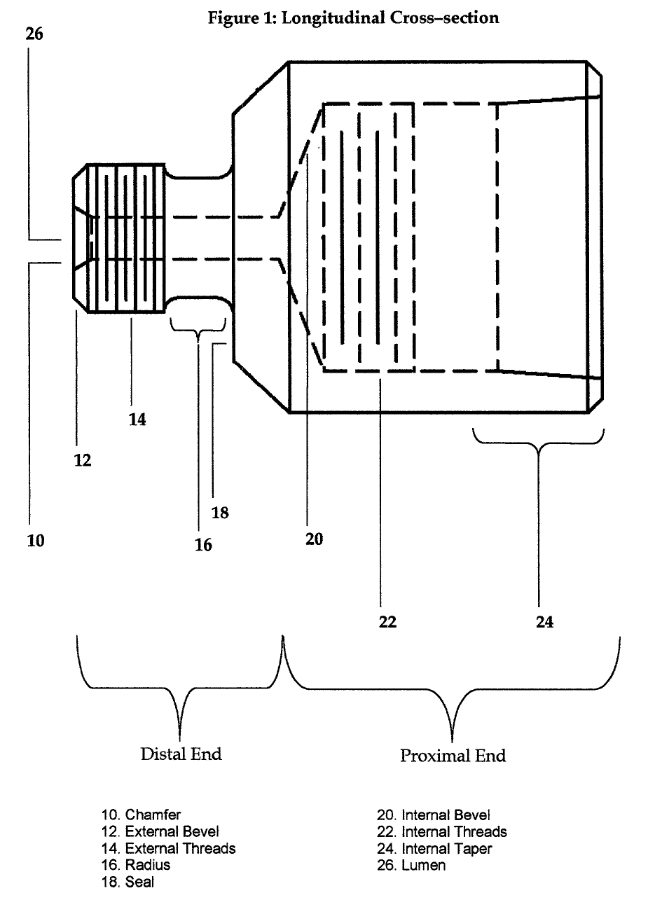

### REFERENCE NUMERALS IN FIG. 1

10. Chamfer

12. External Bevel

14. External Threads

16. Radius

18. Seal

20. Internal Bevel

22. Internal Threads

24. Internal Taper

26. Lumen

### Description—FIG. 1

The invention is one physical piece consisting of two functional units: the proximal end for securing it to the pen syringe and the distal end for securing a double-ended needle to it.

The distal end has external threads 14, which correspond to the internal threads of the double-ended needle hub. It has a lumen 26, which permits the passage of the proximal end of the double-ended needle; a chamfer 10, which guides the proximal end of the double-ended needle through the lumen in a way that will cause the needle to puncture the seal on the distal end of the pen syringe, and not collide with the metal band that holds the seal onto the medication cartridge of the pen syringe. The distal end also has a radius 16, that permits full seating of the double-ended needle to the external bevel 12 and seal 18, which prevents loss of pressure during the injection.

The proximal end has an internal taper 24, which guides the internal threads 22 onto the external threads of the distal end of the pen syringe in the correct alignment and prevents cross threading. The proximal end also has an internal bevel 20, which acts as a stop and seals the connection between the invention and the pen syringe, preventing loss of pressure during the injection.

A typical embodiment of the invention is to be machined from medical-grade polycarbonate rod. It can also be made of any other thermoplastic, moldable or machine-able plastic or metal, such as polyethylene or stairless steel.

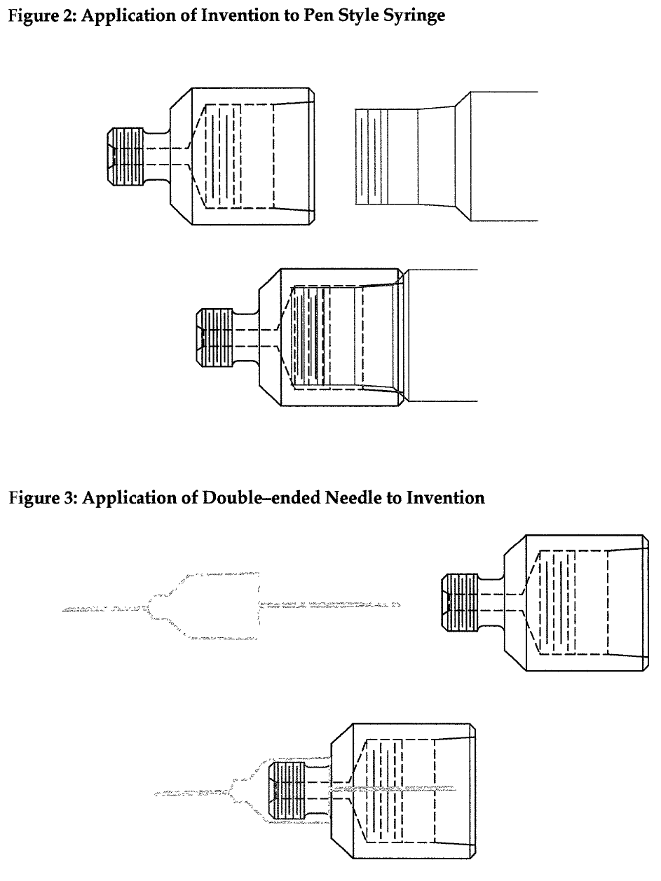

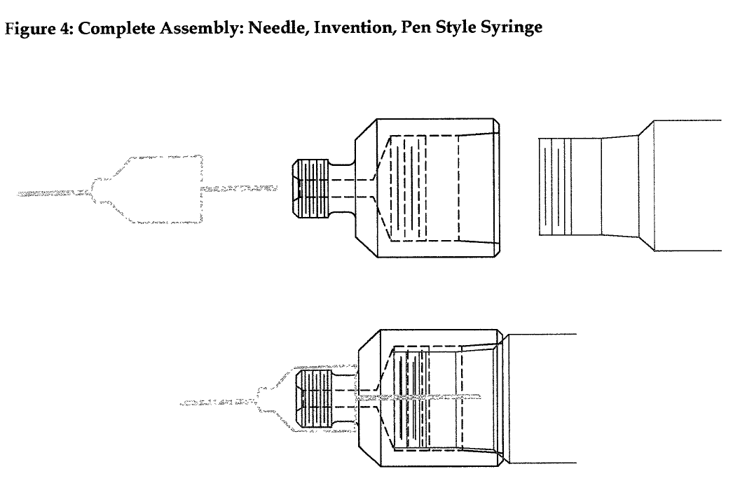

### Operation—FIGS. 2 to 4

The manner of using the adapter is as follows:

The proximal end of the adapter is threaded in a clockwise manner onto the distal end of the pen syringe for the full length of the threads. It stops when it reaches the end of the threads. A new needle (in its protective cover) is grasped with both hands and the seal is cracked, removing one end of the cover, thus exposing the needle extension, or single-beveled, proximal part of the needle.

This end of the needle is passed through the lumen of the distal end of the adapter and the needle's hub is threaded in a clockwise manner onto the exposed threads of the distal end until it stops. During this process, the proximal end of the double-ended needle pierces the rubber plug in the distal end of the pen syringe, thus allowing the passage of the medication through the needle.

Prior to injecting the medication, the protective cover of the tri-beveled, distal end of the double-ended needle is removed, and the air is expressed from the needle lumen. This step is also done when using needles of the prior art.

When the injection is finished, the double ended needle is removed from the distal end of the adapter and disposed of. The adapter can remain attached to the pen syringe because it fits inside the cap.

If the adapter is removed from the pen syringe while the double ended needle is still attached, it is of suffident size to prevent accidental injury from the proximal end of the needle, which remains recessed inside the proximal end of the adapter.

I claim:

1. An adapter, comprising a means to secure a double-ended needle to a pen style medication delivery syringe whereby such adapter consists of:
a. A thermoplastic, moldable, machineable material which is cylindrical and solid and has an axis, a proximal and a distal end
b. Said cylinder's diameter is no less than 0.375 inches and no greater than 0.625 inches Said cylinder's length is no less than 0.35 inches and no greater than 1 inch
c. Said cylinder has an axially oriented, centrally located lumen with a diameter of no less than 0.015 inches and no more than 0.475 inches comprising the means of directing the needle extension of the double ended needle through the rubber plug that seals the distal end of the pen style syringe without contacting the metal ring that seals the rubber plug onto the medication reservoir of the pen syringe
d. Said lumen has a bevel on said distal end comprising the means to guide the needle extension of the double ended needle through the lumen and into the rubber plug that seals the medication reservoir of the pen style syringe
e. Said cylinder has a proximal end and an internal diameter fitted with internal threads of sufficient dimension to correspond with the external threads and external diameter of the distal end of the pen syringe
f. Said internal threads comprise the means of affixing said proximal end to the distal end of a pen style medication delivery syringe
g. Said internal threads begin at a point no less than 0.150 inches and no more than 0.4 inches from the external surface of said proximal end for the purpose of aligning said cylinder on the distal end of the pen syringe in a manner that prevents cross-threading.
h. Said proximal end has an internal taper of sufficient dimension to correspond with the external taper of the pen style syringe
i. Said cylinder has a distal end fitted with external threads of sufficient dimension to correspond with the internal threads of the double ended needle hub
j. Said external threads comprise the means to secure said distal end to the internal threads of the hub of the double ended needle hub
k. Said distal end has a radius thread relief comprising the means of sealing the threads of the double-ended needle hub to the mating surface of the adapter.

2. An adapter according to claim 1, which is of sufficient size to remain attached to a pen-style syringe when said syringe's protective cap is attached.

3. An adapter according to claim 1 which comprises the means to prevent a needle stick if proximal end of said adapter is removed from said pen style syringe while said double-ended needle is attached.
Said proximal end has a lumen of sufficient depth to be greater than the length of the proximal end of said double-ended needle
